(19) United States
(12) Patent Application Publication
(10) Pub. No.: US 2014/0024670 A1
(43) Pub. Date: Jan. 23, 2014

(54) VETERINARY ANTI-PROLACTIN COMPOSITION FOR RUMINANTS

(71) Applicants: Naomi Isaka, Libourne (FR); Thierry Bertaim, Coutras (FR)

(72) Inventors: Naomi Isaka, Libourne (FR); Thierry Bertaim, Coutras (FR)

(73) Assignee: CEVA SANTE ANIMALE, Libourne (FR)

(21) Appl. No.: 13/985,133

(22) PCT Filed: Feb. 15, 2012
(86) PCT No.: PCT/EP12/52553

§ 371 Date: Oct. 11, 2013

(30) Foreign Application Priority Data
Feb. 15, 2011 (FR) 1151243

(51) Int. Cl. A61K 31/48

(52) U.S. Cl. CPC A61K 31/48
USPC 514/288; 546/69

(57) ABSTRACT

The present invention relates to a veterinary anti-prolactin composition to be administered to ruminants. Said composition includes at least one anti-prolactin compound which is an agonist for dopamine receptors and which is particularly useful for preventing and/or reducing the harmful effects in ruminants linked to use for a shortened dry period. Said composition is particularly useful for preventing and/or reducing metabolic diseases and/or reproductive disorders when lactation is resumed. The use of the veterinary anti-prolactin composition according to the invention does not adversely affect the milk-producing ability and/or the milk quality of the treated ruminants

The present invention relates to a veterinary composition and to a method for preventing and/or reducing the deleterious effects usually observed during the implementation of a shortened dry period in ruminants. These include notably metabolic diseases and reproductive disorders when lactation is resumed. The veterinary compositions and method of prevention according to the invention do not affect the milk-producing ability and/or milk quality of the ruminant during the subsequent lactation.

In ruminants, lactation generally lasts for 5 to 20 months. In dairy cows it is most often 10 months. After this lactation period, milking is stopped, generally suddenly, and the ruminant produces no more milk until calving, when the subsequent lactation begins. This period between the halting of milk removal and the subsequent calving, known as the dry period, is generally set at about 2 months in dairy cows.

Although a dry period is necessary to ensure optimal milk production during the subsequent lactation, this dry period necessitates several dietary changes and transitions to adapt to the physiological needs of an animal that is no longer producing milk. These dietary changes cause numerous physiological disruptions and stresses in ruminants, such as disruptions of the balance of the digestive flora, decreases in stomach capacity and absorption with changes in the size of the rumen and the villi of the ruminal mucosa. Finally, this dry period leads to a metabolism in ruminants that is very different from that observed during lactation. Thus, during the resumption of lactation after calving, the ruminant is in a state of metabolic maladjustment resulting in an energy deficit, whereas the need to produce milk is again at a maximum.

In addition, dietary changes and transitions that impose a long dry period make it more complex to administer such dietary programs to the herd. A simplification of these programs, by shortening the dry period, would facilitate herd management for the stockbreeder.

Attempts to reduce the duration of the dry period have been made in the past in order to decrease the negative consequences described above. For example in cows, dry periods of 1 month instead of the usual 2 months have been tested. Nevertheless, it proved that the practice of shortened dry periods actually worsened metabolic imbalances in ruminants and resulted in an increased risk of the occurrence of metabolic diseases and reproductive disorders in the first months of the subsequent lactation. In addition to this significant harm to the ruminant, these practices generally lead to lower milk production during lactation.

In veterinary medicine, prolactin inhibitor-based compositions such as Galastop® (cabergoline, Ceva Santé Animate) can be administered as treatments for lactation during false pregnancies in bitches. They are also prescribed following early weaning, for abortions, or during immediate removal from the litter after parturition in order to halt milk production and suckling. The French patent no. FR 2 936 710 granted on 7 Jan. 2011 in the name of the Applicant describes the administration of anti-prolactin dopamine receptor agonist compounds to ruminants during gestation in order to induce lactation dry-off and to promote mammary involution.

The Applicant has now discovered, surprisingly, that the administration of a veterinary composition comprising at least one anti-prolactin dopamine receptor agonist compound to ruminants at the time of the implementation of the dry period and/or during the dry period made it possible to prevent and/or reduce the deleterious effects related to the implementation of a shortened dry period, such as metabolic diseases and/or reproductive disorders, and also made it possible to improve the immune status and/or hepatic functions of ruminants during the subsequent lactation. Surprisingly, the administration of the veterinary compositions according to the present invention does not, however, affect the milk-producing ability and/or milk quality of the treated ruminants.

### SUMMARY OF THE INVENTION

The present invention relates to veterinary compositions comprising at least one anti-prolactin dopamine receptor agonist compound administered to ruminants at the time of the implementation of and/or during the dry period. The anti-prolactin compounds of the veterinary compositions according to the invention are selected from dopamine receptor agonist compounds resulting from ergot of rye or from agonist compounds not resulting from derivatives of ergot of rye.

According to the invention, said compositions are administered in effective therapeutic amounts, at the time of the implementation of the dry period and/or during the dry period of ruminants, in order to prevent and/or reduce the deleterious effects related to the implementation of a shortened dry period in ruminants. The deleterious effects aimed at by these treatments comprise notably metabolic diseases and reproductive disorders. The administration of the veterinary compositions according to the present invention in addition makes it possible to improve the immune status and/or the hepatic functions of ruminants when lactation is resumed. The compositions are administered to ruminants at the beginning and/or during the dry period, in a single treatment and/or in repeated treatments. The joint administration of veterinary compositions for reducing the duration of the dry period does not affect the milk-producing ability and/or milk quality of the ruminant during the subsequent lactation.

The present invention also relates to a method of treating and/or preventing the deleterious effects related to the implementation of a shortened dry period in ruminants, such as metabolic diseases and/or reproductive disorders, as well as to a method of improving the immune status and/or hepatic functions in ruminants, comprising the administration according to an effective therapeutic regimen of the veterinary compositions to ruminants at the time of the implementation of and/or during the dry period.

### BRIEF DESCRIPTION OF THE FIGURES

FIG. 1: is a graph showing the annual cycle of the cow on a 12-month cycle.

FIGS. 2A and 2B: are graphs showing the progression of milk production 60 days post-calving (2A), and the relative difference with the historical production (305 days from the preceding lactation) (2B), for the treated groups C646-1 and C646-2.

FIG. 3: is a graph illustrating the induction of the mRNA transcript corresponding to the long form of the prolactin receptor during the dry period and up to 90 days post-calving in the group of cows having received an injection of cabergoline (C646-1 and C646-2).

### DETAILED DESCRIPTION OF THE INVENTION

The present invention relates to a veterinary composition comprising at least one anti-prolactin dopamine receptor agonist compound administered in a single treatment and/or in repeated treatments at the time of the implementation of and/or during the dry period, for use in preventing and/or reducing the deleterious effects related to the implementation of a shortened dry period in ruminants.

The present invention also relates to a veterinary composition comprising at least one anti-prolactin dopamine receptor agonist compound administered in a single treatment and/or in repeated treatments at the time of the implementation of and/or during the dry period, for use in preventing and/or reducing metabolic diseases and/or reproductive disorders of ruminants when lactation is resumed.

When said compositions are administered to ruminants at the time of the implementation of and/or during the dry period, they make it possible to improve the immune status and the hepatic functions of ruminants when lactation is resumed, without however affecting milk-producing ability or milk quality during the subsequent lactation.

The present invention also relates to a method of treating and/or preventing the deleterious effects related to the implementation of a shortened dry period in ruminants, such as metabolic diseases and/or reproductive disorders, as well as to a method of improving the immune status and/or hepatic functions in ruminants, comprising the administration according to an effective therapeutic regimen of the veterinary compositions to ruminants at the time of the implementation of and/or during the dry period. This method does not affect the milk-producing ability and/or milk quality of the ruminant.

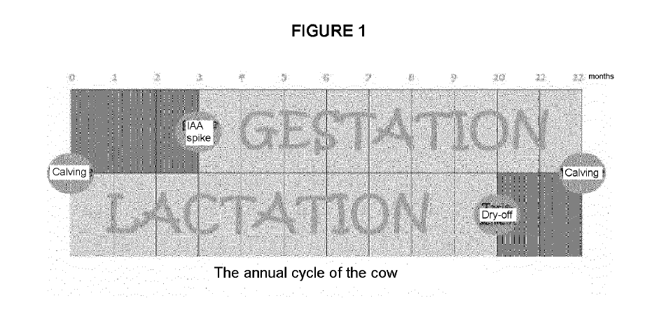

Preferably, said veterinary compositions are administered to ruminants such as dairy cows at the time of the implementation of the dry period, such as shown in FIG. 1.

The veterinary anti-prolactin compositions and the methods according to the present invention are attractive to stockbreeders as their use can decrease the duration of the dry period, resulting in a longer milk production period, simplified herd management, and savings in terms of disease treatment costs when lactation is resumed.

As shown in the examples below, a single treatment and/or repeated treatments can be administered to ruminants at the time of the implementation of and/or during the dry period, in order to prevent and/or reduce metabolic diseases and reproductive disorders, and to improve the immune status and/or hepatic functions of ruminants when lactation is resumed. For example, the veterinary compositions can be administered in dairy cows at the beginning of the dry period or during the dry period.

Conventional dry periods are generally of 8 weeks. Consequently, according to the present invention, the term “shortened dry period” refers to a dry period that is reduced by 1 week, or 2 weeks, or 3 weeks, or 4 weeks. Preferably, the shortened dry period is reduced compared to conventional dry periods by at most 4 weeks, and even more preferentially it is reduced by 2 to 4 weeks. Consequently, according to the present invention, the conventional 8-week dry period is reduced to a shortened dry period whose duration preferably is between 4 to 6 weeks.

The administration of the compositions according to the present invention thus enables:
(i) the prevention and/or reduction of metabolic diseases;
(ii) the prevention and/or reduction of reproductive disorders;
(iii) improved immune status and hepatic functions when lactation is resumed;
(iv) better health of the newborn;
(v) the absence of effects on milk-producing ability and/or milk quality during the subsequent lactation;
(vi) the lengthening of the lactation production period in progress; and
(vii) better management of dietary programs during the dry period.

The anti-prolactin compounds according to the invention are selected from dopamine receptor agonist compounds resulting from ergot of rye and/or from agonist compounds not resulting from derivatives of ergot of rye.

Prolactin is a pituitary hormone with a mammotropic and lactogenic effect, i.e., it activates the growth of mammary glands and the secretion of milk. Prolactin release is under the stimulatory influence of prolactoliberin and the inhibitory influence of dopamine. The inhibitory action of dopamine on the pituitary gland is mediated by post-synaptic dopamine receptors, notably such as the D2 receptor.

Compounds are referred to as “anti-prolactin” when they inhibit the release of prolactin. Dopaminergic compounds are dopamine receptor agonists, able to bind to dopamine receptors present notably on prolactin-secreting cells of the anterior pituitary in order to inhibit the secretion of prolactin.

Preferably, these dopaminergic anti-prolactin compounds bind to dopamine receptors. Still more preferably, these compounds specifically bind to D2 dopamine receptors. Dopaminergic anti-prolactin compounds act by stimulating the release of dopamine in the hypothalamus, thus leading to inhibition of prolactin secretion.

The hormone prolactin binds to membrane receptors (prolactin receptors, PRLr) present on the cells of tissues, notably of the mammary glands, the immune system and liver tissue. PRLr have an extracellular domain which interacts with the hormone, a transmembrane domain, and a cytoplasmic domain involved in transmission of the prolactin signal. The signal is transmitted by the transmembrane translocation of associated tyrosine kinases. There are several isoforms of the prolactin receptor, all of which include a similar extracellular domain but which differ by the length of their transmembrane domain. In bovine species, a long form and short form of the receptor have been described in particular. Prolactin binds to these different receptors with a similar affinity, but transmembrane translocation is promoted by the presence and quantity of the long forms. The co-expression of various isoforms of prolactin receptors (long form versus short form) could modulate the cellular signal due to the prolactin. These anti-prolactin dopamine receptor agonist compounds can be selected from compounds resulting from ergot of rye and/or derivatives thereof. These ergoline derivatives are well-known in the public domain and have the following general structure:

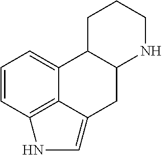 [SMILES: c1cc2c3c(c[nH]c3c1)CC1NCCCC21]

Examples of these anti-prolactin agonist compounds derived from ergot of rye include cabergoline, metergoline, lisurdine, bromocriptine, ergometrine, as well as all derivatives of these compounds having an anti-prolactin activity.

Cabergoline, whose chemical name is N-[3-(dimethylamino)propyl]-N-[(ethylamino)carbonyl]-6-(2-propenyl)-8g-ergoline-8-carboxamide, is an anti-prolactin agonist compound specific for D2 dopamine receptors. It is in particular described in the U.S. Pat. No. 4,526,892. Its chemical structural formula is the following:

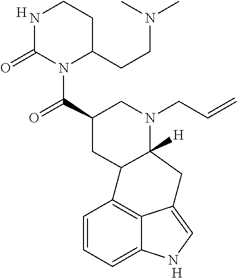

Cabergoline is the active ingredient in human drugs marketed under the names Dostinex® and Cabaser®. Also, it is the basic active ingredient in veterinary compositions marketed under the name Galastop® for bitches prone to lactations of false pregnancy. Neither of these cabergoline-based compositions, Dostinex® or Galastop®, is administered during pregnancy or gestation.

Metergoline is a synthetic compound with the chemical name benzyl ((6aR,9S,10aR)-4,7-dimethyl-4,6,6a,7,8,9,10,10a-octahydroindolo-[4,3-fg]quinolin-9-yl)methyl carbamate. It is an anti-prolactin compound that is able to bind to both dopamine receptors and 5-HT serotonin receptors and thus to activate the release of dopamine and the inhibition of prolactin secretion. Metergoline has the following chemical structural formula:

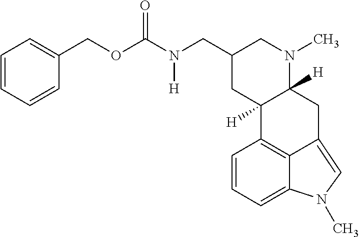 [SMILES: CN1CC(CNC(=O)OCc2ccccc2)C[C@@H]2c3cccc4c3c(cn4C)C[C@H]21]

Lisuride, also known by the chemical name 1,1-diethyl-3-((6aR,9S)-7-methyl-4,6,6a,7,8,9-hexahydroindolo[4,3-fg]quinolin-9-yl)urea, has the following chemical structural formula:

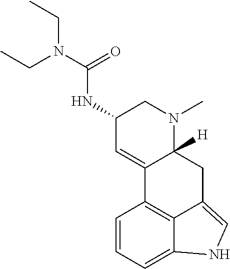 [SMILES: CCN(CC)C(=O)N[C@H]1C=C2c3cccc4[nH]cc(c34)C[C@H]2N(C)C1]

This is a prolactin-inhibiting dopaminergic agonist compound. It is marketed under the name Arolac® for the treatment of amenorrhea and hyperprolactinemia in humans.

Bromocriptine is known by the name bromo-2 ergocryptine and by the chemical name ergotaman-3′,6′,18-trione, 2-bromo-12′-hydroxy-2′-(1-methylethyl)-5′alpha-(2-methylpropyl). Its chemical structural formula is the following:

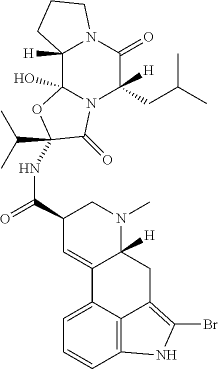 [SMILES: CC(C)C[C@H]1C(=O)N2CCC[C@H]2[C@]2(O)O[C@](NC(=O)[C@@H]3C=C4c5cccc6[nH]c(Br)c(c56)C[C@H]4N(C)C3)(C(C)C)C(=O)N12]

Compositions such as Parlodel® and Bromo-Kin®, used notably to treat pituitary gland tumors and hyperprolactinemia, or in gynecology as a lactation inhibitor after childbirth, comprise a therapeutic amount of bromocriptine, which is a rye ergot derivative and dopaminergic agonist.

Ergometrine, also known as ergonovine and d-lysergic acid beta-propanolamide, can also be incorporated into the veterinary compositions as a dopaminergic anti-prolactin agonist. Its structural formula is the following:

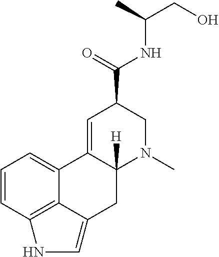

Other anti-prolactin dopamine receptor agonist compounds can also be used in the compositions according to the present invention and can be administered to ruminants particularly during the gestation period. They can be selected from anti-prolactin dopaminergic agonist compounds not resulting from ergot of rye. Examples of these compounds include ropinirole, pramipexole, rotigotine, quinagolide, and all derivatives of these compounds having an anti-prolactin activity.

Ropinirole, whose chemical name is 4-(2-dipropylaminoethyl)-1,3-dihydroindol-2-one, acts as a D2 and D3 dopamine receptor agonist. Its chemical structural formula is the following:

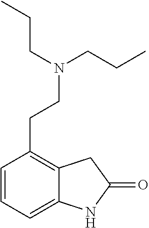 [SMILES: CCCN(CCC)CCc1cccc2c1CC(=O)N2]

Ropinirole is part of the composition of the products Requip® and Ropark® prescribed in human medicine to treat Parkinson's disease.

Pramipexole is another dopaminergic agonist which binds more particularly to D2 and D3 dopamine receptors. Its chemical name is (6S)—N⁶-propyl-4,5,6,7-tetrahydro-1,3-benzothiazole-2,6-diamine and its structural formula is the following:

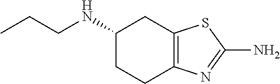

Pramipexole is well-known in the field of human pharmaceutics as it is marketed under the names Mirapex®, Mirapexin® or Sifrol® for the treatment of Parkinson's disease and of restless legs syndrome.

Rotigotine is also known by the chemical name 6-(propyl-(2-thiophen-2-ylethyl)amino)tetralin-1-ol. Its chemical structural formula is the following:

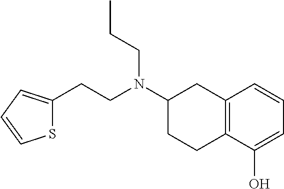 [SMILES: CCCN(CCc1cccs1)C1CCc2c(O)cccc2C1]

Rotigotine was recently approved for the treatment of Parkinson's disease in transdermal administration in the form of patch under the name Neupro®.

Quinagolide, or (3R,4aR,10aS)-3-diethylsulfamoyl amino)-6-hydroxy-1-propyl-3,4,4a,5,10,10a-hexahydro-2H-benzo[g]quinoline, is also an inhibitor of prolactin secretion. It is a specific D2 dopamine receptor agonist. Its chemical structural formula is the following:

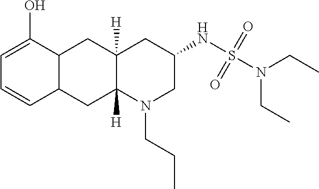 [SMILES: CCCN1C[C@@H](NS(=O)(=O)N(CC)CC)C[C@@H]2CC3C(O)=CC=CC3C[C@H]21]

Quinagolide is marketed under the trade name Norprolac® (Ferring Pharmaceuticals) for the treatment of macroprolactinoma or hyperprolactinemia.

The veterinary compositions can be administered as a single treatment and/or repeated treatments at the time of the implementation of and/or during the dry period.

The effective amounts or therapeutic doses are likely to vary according to the ruminants to be treated and according to the mode of administration of the compositions. The dosages, also called therapeutic regimens, can easily be determined by systematic tests on the basis of the examples below and are within the ability of persons skilled in the art. For example, the effective therapeutic doses according to the present invention are between 1 and 100 μg/kg, or between 5 and 50 μg/kg, and preferably about 8 to 10 μg/kg.

In the case of dairy cows, it is possible to administer the compositions according to the invention at the time of the implementation of and/or during the dry period, for example after the first 11 months of the cow's annual cycle (FIG. 1).

According to the invention, the term “ruminants” refers to herbivorous mammals such as, for example, bovines, sheep, caprines, camelids or bovids. The compositions according to the invention are administered to milk-producing ruminant mammals, preferably such as dairy cows and goats.

The veterinary compositions can be administered according to all routes of administration well-known in the field and suited to the treatment for each animal. Preferably, they are administered by cutaneous, oral, intramammary and parenteral routes. Still more preferentially, they are administered by parenteral route, and notably by intramuscular or subcutaneous injection. They can thus be in the form of an oral, intramammary or injectable liquid solution or suspension, or in solid or semi-solid form, as powders, tablets, capsules, granules, sugar-coated pills, gelcaps, sprays, cachets, pills, bars, or pastes.

Advantageously, the veterinary compositions can be administered in a single injectable dose or in several injectable doses.

According to the formulations of the compositions used, they can further comprise ingredients conventionally used in pharmacy for the preparation of liquid or solid formulations for oral, intramammary or parenteral administration. Furthermore, in the case of oral formulations, they can be administered directly to ruminants or mixed with food.

Also, the compositions according to the invention can comprise according to the type of formulations, a solvent, a glidant, a lubricant and any excipient of suitable mass, such as lactose, cellulose or starches. Stearic acid, magnesium stearate, L-leucine or, for example, glycerol tribehenate can be used as a lubricant. As disintegrating agent, sodium carboxymethylamidone, cross-linked sodium carboxymethyl cellulose or cross-linked polyvinylpyrrolidone can be used. As glidants, pure silica or colloidal silicon dioxide can be used. Solid oral forms can be in the form of tablets covered with a coating.

Injectable preparations are prepared by mixing effective therapeutic amounts of at least one anti-prolactin compound as described above with a solvent, a pH regulator, a buffer agent, a suspending agent, a solubilizing agent, a stabilizer, a tonicity agent and/or a preservative, and by transforming the mixture into a subcutaneous or intramuscular injection according to a conventional method. As solvent, mention may be made of dimethylsulfoxide (DMSO), oily solvents such as medium-chain C₈-C₁₀ triglycerides, or a mixture of capric acid, caprylic acid and triglycerides such as that marketed under the name Mygliol® 812. As needed, the injectable preparations can be lyophilized according to a conventional method. Examples of suspending agents include methyl cellulose, polysorbate 80, hydroxyethylcellulose, xanthan gum, sodium carboxymethylcellulose and polyethoxylated sorbitan monolaurate. Examples of solubilizing agents include polyoxyethylene hardened castor oil, polysorbate 80, nicotinamide, polyethoxylated sorbitan monolaurate, macrogol and castor oil fatty acid ethyl ester. Furthermore, stabilizers include sodium sulfite, sodium metalsulfite and ether, while preservatives include methyl p-hydroxybenzoate, ethyl p-hydroxybenzoate, sorbic acid, benzyl alcohol, phenol, cresol and chlorocresol. An example of a tonicity agent is mannitol. During the preparation of the injectable solutions or suspensions, it is desirable to make sure that they are isotonic with blood.

The present invention further relates to a kit for veterinary use for preventing and/or reducing the deleterious effects related to the implementation of a shortened dry period in ruminants, such as metabolic diseases and/or reproductive disorders, and to improve immune status and/or hepatic functions when ruminant lactation returns. The kits according to the present invention include at least one compartment for an optionally sterile packaging including an effective therapeutic amount of at least one anti-prolactin dopamine receptor agonist compound as described above, for administration to ruminants. The kit includes the means enabling the administration of the compositions by cutaneous, oral, intramammary or parenteral route as well as instructions relating to the mode of administration of the veterinary compositions or drugs according to the invention.

### EXAMPLES

### Example 1

### Preparation of Cabergoline-Based Veterinary Compositions

Preparation of 9 liters of a preparation for injectable solution containing 500 ng/ml of cabergoline.

### Formula:

Active ingredient: 4.53 g of cabergoline (titer 100%)
Excipient: medium-chain triglycerides q.s. 9 liters.

### Step 1:

4.53 g of cabergoline and 1.5 kg of medium-chain triglycerides were measured in a container of suitable capacity, and then the mixture was stirred using a magnetic stirrer (500 rpm) for at least 60 minutes for complete dissolution.

### Step 2:

In a dry container, 5 kg of medium-chain triglycerides were measured, the nitrogen stream and stirrer turned on, and then cabergoline in concentrated solution, obtained in step 1, was added to the container. Stirring was maintained for 30 minutes and then the volume was brought up to the final volume of 9 liters by adding medium-chain triglycerides. Stirring was maintained for 30 minutes more and then the solution was filtered under pressurized nitrogen through a 0.45 micron cartridge. The filtrate was collected in a suitable flask previously steam-disinfected and dried under a nitrogen stream. The solution obtained was sterilized at 121° C. for 15 minutes and then distributed in sterile apyrogenic bottles and sealed with rubber stoppers and aluminum caps, washed and passed to the autoclave.

### Example 2

### Demonstration of the Absence of Deleterious Effects on Milk Production in Dairy Cows Treated with Cabergoline-Based Veterinary Compositions

This study aims to evaluate the effectiveness of cabergoline-based veterinary compositions in primiparous (n=17) or multiparous (n=54) Prim'Holstein dairy cows, in lactation and at 8 months gestation during the administration, in reducing the duration of the dry period to 1 month. The dairy cows are housed in a cowshed according to current breeding conditions.

The experiment comprises 3 groups of cows:
the control group (n=24 cows) which receives two administrations of placebo at D0 (beginning of the dry period by dry-off) and at D10 (10 days after D0);
the C646-1 group (n=23 cows) which receives a single dose of cabergoline-based veterinary composition at D0;
the C646-2 group (n=24 cows) which receives two doses of cabergoline-based veterinary composition at D0 and then at D10.

The cabergoline-based veterinary composition is an injectable oily cabergoline solution as described in Example 1. It is administered by intramuscular injection in the neck in a dose of 5.6 mg/cow, or about 8 ng/kg.

The three groups of treated cows are included in the study at approximately 45 days before calving (D-15) and are monitored for the following three periods:
at the end of lactation (n−1) preceding D0:
during the dry period of 1 month: from milk dry-off (D0) until calving (C0 corresponding to around D30),
at lactation (n) during the first 3 months following calving (C0).

The effective dry periods of the three groups are listed in Table 1 below:

TABLE 1

| | Effective dry period |
|---|---|
| Group | (standard deviation) |
| Placebo | 31 days (5.0) |
| C646-1 | 27 days (4.8) |
| C646-2 | 29 days (5.4) |

Milk production 3 months after calving (C0) is obtained for each treated cow and is compared to production obtained during the preceding lactation (n−1).

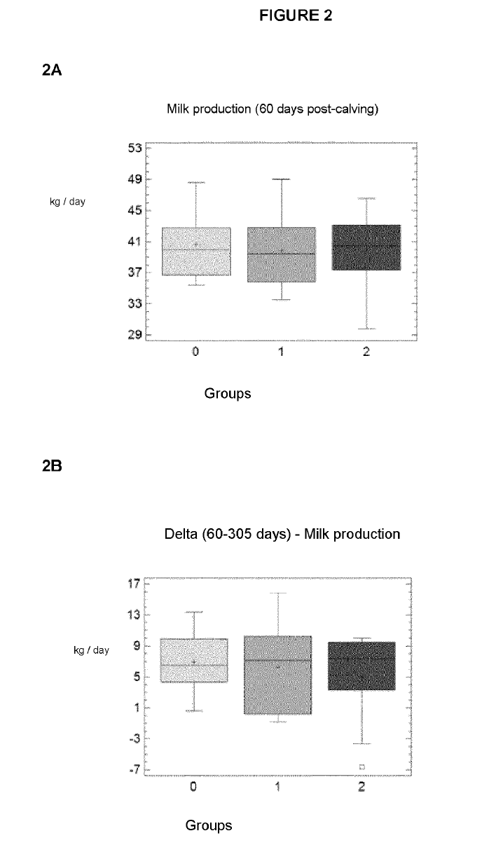

Milk production during the 60 days post-calving (FIG. 2A) as well as the relative difference with the historical production (305 days from the preceding lactation), (FIG. 2B) shows that the duration of the dry period following the treatments with the veterinary compositions according to the present application does not have deleterious effects on the milk-producing ability of the treated dairy cows.

### Example 3

### Study of the Effectiveness in Dairy Cows of Cabergoline-Based Veterinary Compositions

The three groups of cows treated as described in Example 2 undergo the following examinations and samplings:
Daily general clinical monitoring:
   Appearance of diseases (metabolic, infectious, etc.);
   All abnormal signs are noted, notably such as abortions, as well as any difficulties during calving. The animals are followed until being dropped in order to evaluate the state of health of the newborn calves.
Blood samples are taken at D0, D10, D20, C0 (calving=D30), C10, C30 and C90.
   These samples enable us to assay by PCR notably the prolactin receptors considered to be markers of animal health,
Colostrum (IgG) assay at C0
Counts of somatic cells in milk produced at C30, C60 and C90.

The results observed reveal that the reduction of the duration of the dry period is expressed notably by increased expression of the long form of prolactin receptors in the C646-1 and C646-2 groups compared to the placebo group.

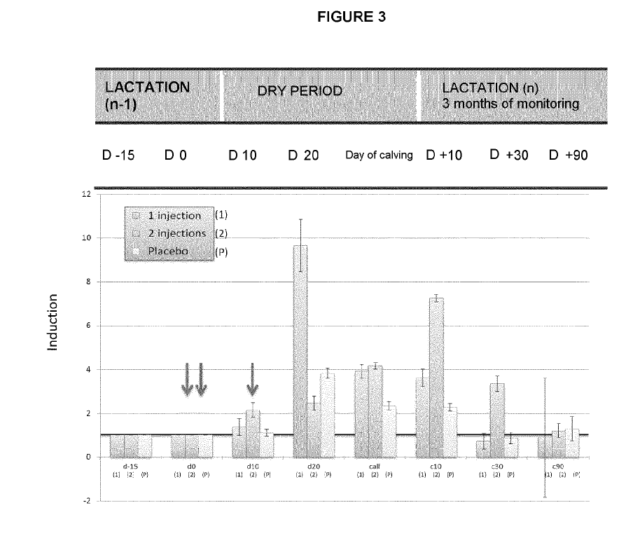

FIG. 3 indeed shows an induction of the expression of the long isoform of the prolactin receptor during the dry period and at 10 days and then 30 days post-calving in the group of cows having received an injection of a veterinary composition according to the present invention.

1.-19. (canceled)

20. A method for preventing and/or reducing the deleterious effects related to the implementation of a shortened dry period in ruminants, wherein said method comprises administration of a veterinary composition comprising at least one compound having an anti-prolactin dopamine receptor agonist activity, wherein said deleterious effects are selected in the group consisting of metabolic diseases, reproductive disorders, immune status or hepatic functions disorders when lactation is resumed, and bad health of the newborn.

21. The method according to claim 20, wherein said method is for preventing and/or reducing metabolic diseases and/or reproductive disorders when lactation is resumed.

22. A method for a decrease of the dry period of ruminants by administering to a ruminant a veterinary composition comprising at least one compound having an anti-prolactin dopamine receptor agonist activity.

23. The method according to claim 20, wherein the composition is administered to the ruminant at the time of the implementation of and/or during the dry period.

24. The method according to claim 20, wherein the composition is administered in a single treatment and/or in repeated treatments.

25. The method according to claim 20, wherein the composition is administered at doses from about 8 to 10 μg/kg.

26. The method according to claim 20, wherein the ruminants are herbivorous mammals selected in the group consisting of bovines, sheep, caprines, camelids and bovids, more preferably dairy cows.

27. The method according to claim 20, wherein the dry period is a 8-week dry period that is reduced to a shortened dry period of between 4 and 7 weeks, or between 4 and 6 weeks, or between 4 and 5 weeks, and preferably between 4 and 6 weeks.

28. The method according to claim 20, wherein the dry period is reduced by 2 to 4 weeks.

29. The method according to claim 20, wherein the milk-producing ability and/or milk quality of the ruminant is not affected.

30. The method according to claim 20, wherein the dopamine receptor agonist compound is selected from agonists resulting from ergot of rye and/or derivatives thereof.

31. The method according to claim 20, wherein the dopamine receptor agonist compound is selected from cabergoline, metergoline, lisurdine, bromocriptine, ergometrine, ropinirole, pramipexole, rotigotine, quinagolide, and/or a derivative thereof.

32. The composition according to claim 20, wherein said composition is administered by parenteral, intramammary, cutaneous or oral route.

33. A kit including a veterinary composition comprising at least one compound having an anti-prolactin dopamine receptor agonist activity in an effective therapeutic dose, for use in preventing and/or reducing the deleterious effects related to the implementation of a shortened dry period in ruminants, the said deleterious effects are selected in the group consisting of metabolic diseases, reproductive disorders, immune status or hepatic functions disorders when lactation is resumed, and bad health of the newborn.

34. The kit according to claim 33, wherein said composition is administered to ruminants at the time of the implementation of and/or during the dry period.
